(19) United States
(12) Patent Application Publication
(10) Pub. No.: US 2026/0306872 A1
(43) Pub. Date: Oct. 8, 2026

(54) DEVICE AND METHOD FOR TREATING DRY EYES

(71) Applicant: Lumenis Be Ltd., Yokneam (IL)

(72) Inventors: May Kleinman Ben Tsvi, Binyamina (IL); Yosef Birman, Haifa (IL)

(21) Appl. No.: 19/481,597

(22) PCT Filed: May 2, 2024
(86) PCT No.: PCT/IL2024/050419

§ 371 Date: Nov. 5, 2025

Related U.S. Application Data
(60) Provisional application No. 63/464,847, filed on May 8, 2023.

(51) Int. Cl. A61N 5/06; A61B 17/00; A61B 18/00; A61F 9/00; A61N 1/40

(52) U.S. Cl. CPC A61N 5/0624; A61F 9/00; A61N 1/40; A61B 2017/00154; A61B 2017/0023; A61B 2017/00473; A61B 2017/00907; A61B 2018/00994; A61N 2005/063; A61N 2005/0644

(57) ABSTRACT

A device and method for treating eyelid or per-ocular area of a patient. The device has an optical transmission assembly having a proximal end and a distal end, the proximal end of the optical transmission assembly being connectable to an optical light source for receiving intense pulsed light (IPL) therefrom; a radio-frequency (RF) transmission assembly having a proximal end and a distal end, the proximal end of the RF transmission assembly being connectable to a RF energy source for receiving RF energy therefrom; a handpiece having a proximal end and a distal end, the handpiece mounted around at least a distal side of the optical transmission assembly; a first removable tip removably connectable to the distal end of the handpiece and configured to be positioned in contact with the eyelid or peri-ocular area of the patient to transmit the intense pulsed light (IPL) from the distal end of the optical transmission assembly to the eyelid or peri-ocular area of the patient; and a second removable tip removably connectable to the distal end of the handpiece and configured to be positioned in contact with the eyelid or peri-ocular area of the patient to transmit the RF energy from the distal end of the RF transmission assembly to the eyelid or peri-ocular area of the patient.

### RELATED APPLICATIONS

This application is a continuation of priority filing U.S. Provisional Application No. 63/464,847 filed May 8, 2023, entitled “Device and Method for Treating Dry” the entire contents of which are herein incorporated by reference.

### TECHNICAL FIELD

The present disclosure relates to a medical device. Particularly but not exclusively, the present disclosure relates to a device and a method for treating dry eyes.

### BACKGROUND

One reason for dry eyes is Blepharitis, a chronic inflammation of the eyelid margin. If eyelid glands are chronically inflamed, they produce too little or even no oil, which leads to an unstable tear film. The treatment of chronic Blepharitis is not trivial, as it is often caused by pathogens such as bacteria and skin mites (demodex). When one suffers from dry eye due to MGD (Meibomian Glands Dysfunction), the Meibomian glands may be clogged, which may lead to dysfunction of the Meibomian glands, i.e., not producing the necessary oil required to control the tears evaporation rate.

It has been shown that, for example, treatment with IPL (Intense Pulsed Light) therapy has a long-lasting effect and bacteria and skin mites are successfully eliminated, and the inflammation cycle stopped. IPL is a light pulse therapy that may be used to treat the inflammatory causes of dry eye, stimulate the eyelid glands, and accelerate healing. Other light sources, such as LED or laser light, may be used. The use of a light source is for the purpose of generating heat, and causing photo modulation, so as to heat up the area of a Meibomian Gland, softening the paste-like oil to a more liquid form to enable easy expression of the glands. Other sources of generating heat may be implemented to provide treatment.

U.S. Pat. Nos. 9,333,370 and 10,085,814, assigned to the assignee of the present disclosure and incorporated herein by reference in their entirety, are directed to dry eye treatments using light.

During treatment, several pulses of light may be delivered to the patient's eyelids or to the periocular area surrounding the eyelids. Care should be taken to avoid direct light entry into the cornea, as well as exposure of eyelashes to the energy (to avoid hair loss). In addition, direct heating of the cornea and other ocular surfaces should be avoided.

### SUMMARY

The presently disclosed subject matter provides a system and method for dry eye treatment. IPL treatment on the peri-ocular area may treat MGD inflammation, it requires a few sessions (3-5) to get the maximum effect. Radio-frequency (RF) is another effective way to treat dry eye and is complimentary to the IPL treatments. Meibomian glands may be clogged even with IPL treatment and result in discomfort to the patient. Heating the area, such as with radio-frequency (RF), may help expand the glands, and liquefy the pasty oil, thus enabling to perform expression and may result in immediate relief.

In an embodiment, the present disclosure discloses a device for treating an eyelid of a subject. The device includes a radio-frequency (RF) transmission assembly having a proximal end and a distal end with the proximal end of the RF transmission assembly being connectable to a RF energy source for receiving RF energy therefrom. The device has an optical transmission assembly having a proximal end and a distal end with the proximal end of the optical transmission assembly being connectable to an optical light source for receiving intense pulsed light (IPL) therefrom. There is a handpiece having a proximal end and a distal end and the handpiece is mounted at least a distal end of the optical transmission assembly. The device further includes a first tip (may also be referred to herein as an IPL tip) for delivering IPL energy and removably connectable to the distal end of the handpiece and configured to be positioned in contact with the eyelid of the subject to transmit the intense pulsed light (IPL) from the distal end of the optical transmission assembly to the eyelid of the subject. The device also includes a second tip (may also be referred to herein as a RF tip) for delivering RF energy and removably connectable to the distal end of the handpiece and configured to be positioned in contact with the eyelid of the subject to transmit the RF energy from the distal end of the RF transmission assembly to the eyelid of the subject. Each one of the first (IPL) and second (RF) tips can be configured as a reusable tip. Alternatively, each one of the first and second tips can be configured as a disposable tip, to facilitate sterility management.

In another embodiment, the present disclosure discloses a method of treating dry eye of a subject by the device as described above. The method comprises connecting the first tip to the distal end of the handpiece and performing dry eye treatment by moving the first tip over the eyelids of the subject, preferably with minimum overlap of the tip position between movements while activating the emission of the IPL energy, disconnecting the first tip and connecting the second tip to the distal end of the handpiece and massaging eyelids of the subject with the second tip for a radio frequency (RF) treatment.

In one aspect of the current disclosure there is a device for treating at least one of an eyelid or a per-ocular area of a patient, the device comprising: an optical transmission assembly having a proximal end and a distal end, the proximal end of the optical transmission assembly being connectable to an optical light source for receiving intense pulsed light (IPL) therefrom; a radio-frequency (RF) transmission assembly having a proximal end and a distal end, the proximal end of the RF transmission assembly being connectable to a RF energy source for receiving RF energy therefrom; a handpiece having a proximal end and a distal end, the handpiece mounted around at least a distal side of the optical transmission assembly; a first removable tip removably connectable to the distal end of the handpiece and configured to be positioned in contact with the eyelid or peri-ocular area of the patient to transmit intense pulsed light (IPL) from the distal end of the optical transmission assembly to the eyelid or peri-ocular area of the patient; and a second removable tip removably connectable to the distal end of the handpiece and configured to be positioned in contact with the eyelid or peri-ocular area of the patient to transmit the RF energy from the distal end of the RF transmission assembly to the eyelid or peri-ocular area of the patient. The optical transmission assembly comprises an optical fiber bundle, and the second removable tip includes a plurality of RF electrodes to transmit the RF energy to the eyelid or the peri-ocular area of the patient. Also, at least a portion of the RF transmission assembly extends externally of a cover member of the handpiece.

In another aspect, the device has the RF energy source located in at least one of: inside the handpiece or external to the handpiece and wherein at least one of the first removable tip and the second removable tip is made of bio-compatible material(s). At least one of the first removable tip and the second removable tip is configured to be disposable, and the first removable tip and the second removable tip are defined with a plurality of notches to facilitate assembly with the distal end of the handpiece. The device is also operable for treatment of dry eye syndrome.

In yet another aspect of the disclosure, there is a method of treating dry eye syndrome of a patient, the method comprising: providing a handpiece having a proximal end and a distal end;
providing a first and a second removable tip configured to be removably connectable to the distal end of the handpiece and configured to be positioned in contact with an eyelid or peri-ocular area of the patient; connecting the first removable tip to the distal end of the handpiece; contacting the eyelid or peri-ocular area of the patient with the first removable tip; transmitting an intense pulse light (IPL) received from the proximal end of the handpiece to the distal end to treat the eyelid or peri-ocular area of the patient; replacing the first removable tip with the second removable tip; contacting the eyelid or peri-ocular area of the patient with the second removable tip; transmitting radio-frequency (RF) energy from the proximal end of the handpiece to the distal end to treat the eyelid or peri-ocular area of the patient. The method further comprises massaging the eyelid with the second tip during application the RF treatment; and performing expression on upper and lower eyelids of the eye after applying the RF treatment.

In a final aspect, the method comprises placing an eye shield on the eyeball under the eyelids, to protect the eye, prior to applying the IPL treatment and the second removable tip includes a plurality of RF electrodes to transmit RF energy to the eyelid or the peri-ocular area of the patient. The method is operable for treatment of dry eye syndrome. Transmitting of the IPL is done through a fiber bundle optical transmission assembly, and an RF energy source located in at least one of: within the handpiece or external to the handpiece. Also, at least one of the first removable tip and the second removable tip is made of bio-compatible material(s), and at least one of the first removable tip and the second removable tip is disposable.

### BRIEF DESCRIPTION OF THE ACCOMPANYING DRAWINGS

The novel features and characteristic of the disclosure are set forth in the appended claims. The disclosure itself, however, as well as a preferred mode of use, further objectives, and advantages thereof, will best be understood by reference to the following detailed description of an illustrative embodiment when read in conjunction with the accompanying figures. One or more embodiments are now described, by way of example only, with reference to the accompanying figures wherein like reference numerals represent like elements and in which:

FIG. 1 illustrates a schematic view of a device used for treating eyelid of a subject, or/and peri-ocular area in vicinity of the eyelid, in accordance with an embodiment of the present disclosure.

FIG. 2 illustrates a perspective view of the device of FIG. 1 used for treating eyelid or/and peri-ocular area of the subject using a first removable tip for optical treatment, in accordance with an embodiment of the present disclosure.

FIGS. 3a and 3b illustrate perspective views of the device of FIG. 1 used for treating eyelid of the subject with wired connectors using a second removable tip for radio frequency treatment, in accordance with an embodiment of the present disclosure.

FIGS. 4a and 4b illustrate perspective views of the device of FIG. 1 used for treating eyelid of the subject using a second removable tip for radio frequency treatment, in accordance with an embodiment of the present disclosure.

FIG. 5 illustrates a flowchart of a method of treating dry eye of a subject using the device of FIG. 1, in accordance with an embodiment of the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION

The foregoing has broadly outlined the features and technical advantages of the present disclosure in order that the detailed description that follows may be better understood. It should be appreciated by those skilled in the art that the conception and specific embodiments disclosed may be readily utilized as a basis for modifying or designing other structures for carrying out the same purposes of the present disclosure. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

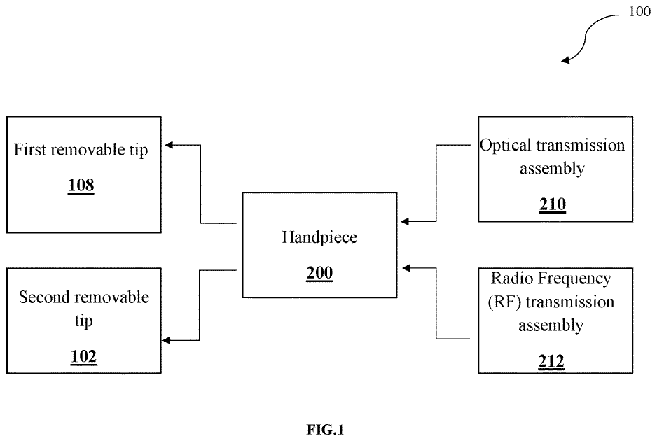

FIG. 1 shows a schematic view of a non-limiting example of a device (100) for treating an eyelid or/and peri-ocular area of a subject (herein after, whenever the text refers to eyelid it should be understood that it also refers to the peri-ocular area in the additive or alternative manner), in accordance with the present disclosure. The device (100) may be used to treat a dry eye syndrome of the subject. In an embodiment, the subject is a human being and device (100) is used to treat dry eye condition of the human being. The device (100) includes a handpiece (200) used to deliver the treatment, and optionally massage the eyelids for treating the dry eye condition of the subject. The device (100) includes an optical transmission assembly (210) inside of the handpiece (200), and a radio frequency (RF) transmission assembly (212). Each of the optical transmission assembly (210) and the RF transmission assembly (212) may be defined with a proximal end and a distal end (not shown in FIG. 1 but will be shown in subsequent figures). Further, the handpiece (200) may selectively be coupled to either an optical light source (not shown) to transmit light energy through the optical transmission assembly (210) or an RF source (not shown) to transmit RF energy through the RF transmission assembly (212). In some embodiments, a portion of the RF transmission assembly extends externally on the cover member of the handpiece.

In an embodiment, a proximal end of the handpiece (200) may be connected to the RF energy source or the optical light source. In some embodiments, the optical transmission assembly may be a fiber optic cable or fiber bundle. In some embodiments, the RF energy source is in the handpiece. In some embodiments, the handpiece RF transmission assembly is connectable to an RF energy source for receiving RF energy from outside the handpiece.

Further, a distal end of the handpiece (200) may be selectively connected to either a first removable tip (108) or a second removable tip (102). In an embodiment, when the handpiece (200) is connected to the optical light source at its proximal end, the handpiece (200) is connected to the first removable tip (108) at its distal end and the optical light energy is transmitted to the eyelid through the optical transmission assembly (210) and the first removable tip (108). Further, when the handpiece (200) is connected to the RF energy source at its proximal end, the handpiece (200) is connected to the second removable tip (102) at its distal end and the RF energy is transmitted to the eyelid through the RF transmission assembly (212) and the second removable tip (102). It is noted that, as mentioned above, each one of the first and second tips can be configured to be a reusable or a disposable tip, where the latter option is preferable from sterility and comfort of usage points of view.

The second removable tip (102) can be used to carry out the RF treatment to warm the peri-ocular area of eyelids before or after IPL treatment. The RF tip, using the same handpiece for treatment, allows for easy access and full visualization of the treated area, enabling the user to perform expression of the meibomian glands in a safer and less painful way to the patient, while inducing immediate or rapid relief.

In an embodiment, the first removable tip (108) and the second removable tip (102) may be made of a bio-compatible material. Further each of the first removable tip (108) and the second removable tip (102) may be ended with a suitable interface, such as notches (104c), at a proximal side thereof to allow assembly of the removable tips with the distal end of the handpiece (200). The first and second removable tips can also be configured with a distal surface that enables effective contact with the eyelid and peri-ocular surfaces.

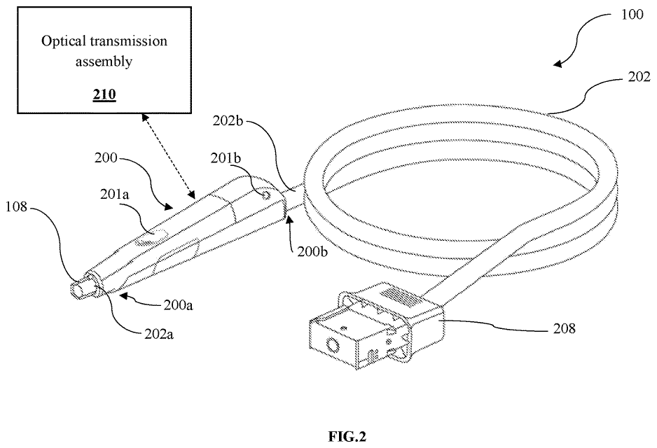

Referring now to FIG. 2, illustrating a non-limiting example of the device (100) with the first, optical, removable tip (108) attached for delivering optical treatment to the eyelid or peri-ocular area. The handpiece (200) is configured for connecting to an optical light source at a proximal end (200b) of the handpiece (200) to transmit optical light energy through the optical transmission assembly (210). Further, the handpiece (200) is also connected to the first tip (108) at a distal end (200a) of the handpiece (200) for carrying out dry eye optical treatment. The proximal end (202b) and the distal end (202a) of the optical transmission assembly (210) are shown. In some embodiments, the proximal end of the optical transmission assembly (210) may be connected to the proximal end (200b) of the handpiece (200) and the distal end of the optical transmission assembly (210) may be connected to the distal end (200a) of the handpiece (200). In some embodiments, a distal side of the optical transmission assembly (210) proximal to the distal end of the optical transmission assembly (210) may be configured to receive the handpiece (200) such that the handpiece (200) surrounds the optical transmission assembly (210) at least at a distal side thereof. In an embodiment, the optical transmission assembly may be a fiber or fiber bundle that is connectable to the optical light source (not shown) such as, but not limited to, Diode Laser. In an embodiment, the optical light source may be an Intense Pulsed Light (IPL) source to carry out IPL treatment of the dry eye of the subject.

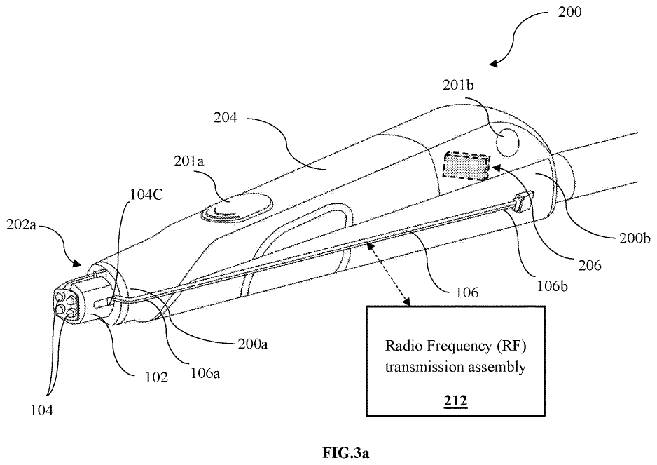

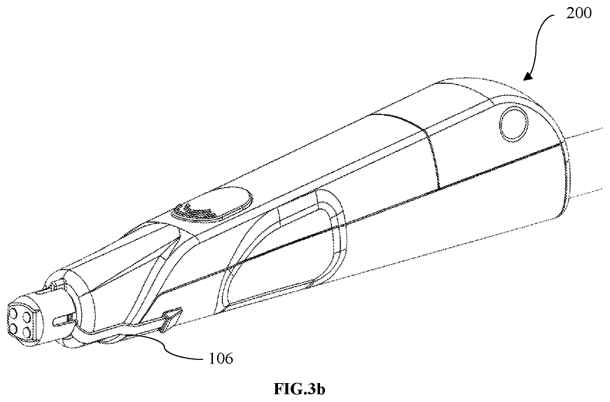

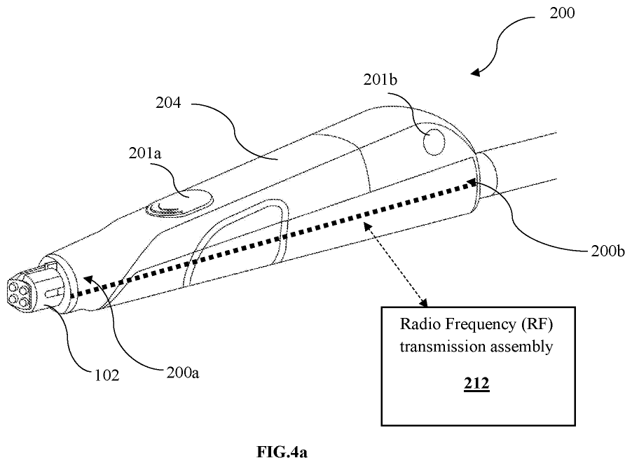

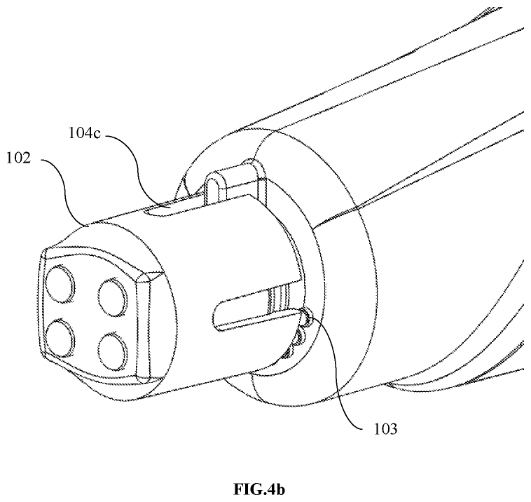

As also shown in FIG. 2, there may be a cord (202) extending proximally from the handpiece and along which signals from the optical light source and possibly also from the RF energy source are carried towards the optical transmission assembly (210) and the RF transmission assembly (212—shown in FIGS. 3a, 3b, 4a and 4b). In some embodiments, at least proximal sides of the optical transmission assembly (210) and the RF transmission assembly (212) pass through the cord (202). A plug (208) may be provided at the proximal end of the cord (202) to connect the device (100) to the optical light source and/or the RF energy source. In an embodiment, the proximal end of the optical transmission assembly (210) extends to the plug (208) connectable to the optical light source.

In some embodiments, the cover member (204) may be mounted around rigid tube (not shown) such that the cover member is freely rotatable relative to the rigid tube. This free rotation of the cover member allows the operator to change the orientation of the device (200) during IPL treatment as may be necessary and thus facilitates better treatment of dry eyes of a human being. The rigid tube about which the cover member is freely rotatable is further described in U.S. application Ser. No. 17/565,747 filed Dec. 30, 2021, and is hereby incorporated by reference in its entirety.

In an embodiment, the handpiece (200) includes a rigid tube (not shown) that houses a portion of the distal side of the optical fiber or fiber bundle of the optical transmission assembly. The handpiece (200) further includes a housing or a cover member (204) (shown in FIG. 3) that is mounted around the rigid tube. In an embodiment, the cover member (204) may be made of materials such as, but not limited to, polymeric or plastic material. The cover member (204) allows a medical practitioner to handle the handpiece (200) of the device (100) while treating the dry eye syndrome of the subject. The cover member (204) can be held through a hand of the medical practitioner and may be used to direct the light energy and the RF energy from respectively the light source and the radio frequency (RF) energy source to the eyelid of the subject. As also show, the handpiece (200) may include a trigger button (201a) to enable the IPL pulse or other energy emission used for the treatment, and an On/Off button (201b) to control the RF energy source.

During the IPL treatment, a light beam from the IPL light source may be transmitted, via the cord (202), from the proximal end to the distal end of the optical transmission assembly and then into the first tip (108). The first tip (108) transmits the light beam via a contact surface to the patient's eyelid or the peri-ocular area. A rounded, solid or soft tip allows a comfortable interface for the physical contact between the first tip (108) and the eyelids/peri-ocular area. The contact surface of the first tip (108) may also be curved so that, when contacting the treated area, the softness of the material distributes the force and the pressure on the treated area. In some embodiments, the contact surface of the first tip (108) may have a concave shape which enables better conformation to the natural curvature of the eyelid over the eyeball.

Referring now to FIGS. 3a and 3b, illustrating two non-limiting examples of the device (100) with the second, RF, removable tip (102) attached at the distal end (200a) of the handpiece for delivering RF treatment to the eyelid/peri-ocular area from an RF energy source. The RF energy source may be located in or connected at the proximal end (200b) of the handpiece (200) to transmit RF energy through the RF transmission assembly (212). The handpiece (200) is connected to the second removable tip (108) at a distal end (200a) of the handpiece (200) for carrying out dry eye RF treatment. In some embodiment, as shown in FIG. 3a, at least a distal portion of the RF transmission assembly (212), in the form of a wired connector (106), is attached externally to the housing (204) of the handpiece (200) to connect, at its proximal end (106b), to an RF energy source (206), located at the proximal side of the device (100) or inside the handpiece (200) and, at its distal end (106a), to the second removable tip (102). In some embodiments, and as shown in FIG. 3b, there is a flat wired connector (106) attached externally to the housing (204) of the handpiece (200) to connect, a RF energy source (206) inside the handpiece (200). In some embodiments, a battery pack (not specifically shown) may be located on/in the handpiece (200) at the proximal end (200b), to power the RF energy source (206). As shown, the distal end (106a) of the wired connector (106) is connected to a plurality of RF electrodes (104) provisioned on the second tip (102). The RF energy source is configured to emit RF energy which is used to carry out RF treatment of a dry eye syndrome of the subject.

Referring now to FIG. 4a and FIG. 4b, at least a distal portion of the RF transmission assembly passes inside the housing (204) of the handpiece (200). The RF energy source may be provisioned within the housing (204) of the handpiece (200) or outside of the handpiece (204). The RF energy can be transmitted through a wire harness running inside the housing (204) of the handpiece (200) and forming at least a distal part of the RF transmission assembly (212), to the plurality of RF electrodes (104) of the second tip (102). In some embodiments, the second tip (102) is connected to the RF energy source through the handpiece (200), with electrical contacts (103). In some embodiments, the electrical contacts (103) are pogo pins. The second tip (102) transmits the RF energy via the RF electrodes (104) to the patient's eyelid or peri-ocular area. A rounded, solid or soft tip allows a comfortable interface for the physical contact between the second tip (102) and the treated area.

In some embodiments, the number of RF electrodes (104) may be four, as shown. In some embodiments, the RF electrodes (104) may be in the form of rounded pins that can be used to massage the eyelid/peri-ocular area and deliver the RF energy thereto. In some embodiments, the RF electrodes (104) can be divided into pairs operating in bi-polar mode. In some embodiments, the RF electrodes (104) can be divided into a first electrode with a first polarity and the remaining electrodes with a second polarity. In some embodiments, a first polarity is cycled through the electrodes while the remaining electrodes have a second polarity.

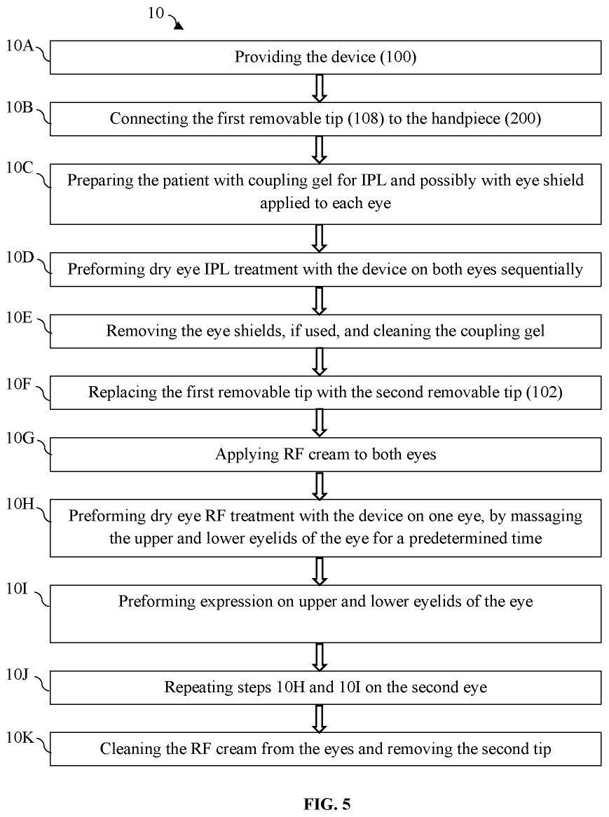

Reference is made to FIG. 5 illustrating a non-limiting example of a method 10 for treating dry eye syndrome. In an embodiment, the method of treating dry eye of the subject utilizes the device (100).

At step 10A, the method includes providing the device (100) including the removable first and second tips. In some embodiments, the first and second tips are disposable as well as removable and are constructed of a bio-compatible, transparent or translucent material, such as silicone or polycarbonate, which allows delivery of light energy IPL to an eyelid. The first and second tips may be provided in clean/sterile state or packaging, for example one per patient or for each eye, to allow hygienic treatment for every patient.

At step 10B, the method includes connecting the first removable tip (108) to the handpiece (200). In some embodiments, the first removable tip is mounted over the distal end of IPL transmission assembly. The mounting may be accomplished by use of an adhesive, or force-fitted (friction between sides of the distal end of IPL transmission assembly and an annular part of the removable tip (not shown)) surrounding the IPL transmission assembly, or snap on and off, or form-fitted (e.g., by a thread). The mounting of a removable tip is further described in U.S. application Ser. No. 17/565,747 filed Dec. 30, 2021, and is hereby incorporated by reference in its entirety.

At step 10C, the method includes preparing the patient with coupling gel for IPL and possibly with an eye shield, preferably applied to each eye. The eye shield is aimed at protecting the eyeball and the internal parts of the eye from the effects of the IPL.

At step 10D, the method includes preforming dry eye IPL treatment with the device on both eyes sequentially.

At step 10E, the method includes removing the eye shields, if used, and cleaning the coupling gel.

At step 10F, the method includes replacing the first removable tip with the second removable tip (102). In some embodiments, the second removable tip is mounted over the distal end of IPL transmission assembly in the same way the first removable tip is mounted. In some embodiments, once the second removable tip is mounted the second removable tip is connected to the RF transmission assembly with electrical contacts (103) or a wired connector (106).

At step 10G, the method includes applying RF cream to both eyes.

At step 10H, the method includes preforming dry eye RF treatment with the device on one eye, by massaging the upper and lower eyelids of the eye for a predetermined period, e.g. a few minutes.

At step 10I, the method includes preforming expression on upper and lower eyelids of the eye.

At step 10J, the method includes repeating steps 10H and 10I on the second eye.

At step 10K, the method includes cleaning the RF cream from the eyes and removing the second tip.

It is appreciated that the sequence of steps above may be altered without departing from the disclosed subject matter. For example, it can be that the sequence of actions can be performed on one eye only or can be performed from start to end on one eye and then repeated on the second eye. Also, it is appreciated that some steps can be repeated before moving to the next step. For example, the RF treatment described in steps 10H and 10I can be repeated several times on each eye, if needed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and are generally intended as “open” terms, e.g. the terms “including” and “includes” should be interpreted as “including/includes but not limited to,” the term “having” should be interpreted as “having at least,”, etc. It will be further understood by those within the art that, as an aid to understanding, the detailed description may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an”, e.g., “a” and/or “an” should typically be interpreted to mean “at least one” or “one or more”; the same holds true for the use of definite articles used to introduce claim recitations. In addition, even if a specific number of an introduced claim recitation is explicitly recited, those skilled in the art will recognize that such recitation should typically be interpreted to mean at least the recited number, e.g., the bare recitation of “two recitations,” without other modifiers, typically means at least two recitations, or two or more recitations.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following detailed description.

### REFERRAL NUMERALS

| Particular | Reference Number |
|---|---|
| Device | 100 |
| Handpiece | 200 |
| Distal end of the handpiece | 200a |
| Proximal end of the handpiece | 200b |
| Cord | 202 |
| Distal end of the optical | 202a |
| transmission assembly | |
| Proximal end of the optical | 202b |
| transmission assembly | |
| Housing/Cover member | 204 |
| Radio frequency (RF) energy | 206 |
| source | |
| Plug | 208 |
| First removable tip | 108 |
| Second removable tip | 102 |
| RF electrodes of the second tip | 104 |
| Connector | 106 |
| Distal end of the RF | 106a |
| transmission assembly | |
| Proximal end of the RF | 106b |
| transmission assembly | |

1. A device for treating at least one of an eyelid or a per-ocular area of a patient, the device comprising:
an optical transmission assembly having a proximal end and a distal end, the proximal end of the optical transmission assembly being connectable to an optical light source for receiving intense pulsed light (IPL) therefrom;
a radio-frequency (RF) transmission assembly having a proximal end and a distal end, the proximal end of the RF transmission assembly being connectable to a RF energy source for receiving RF energy therefrom;
a handpiece having a proximal end and a distal end, the handpiece mounted around at least a distal side of the optical transmission assembly;
a first removable tip removably connectable to the distal end of the handpiece and configured to be positioned in contact with the eyelid or peri-ocular area of the patient to transmit intense pulsed light (IPL) from the distal end of the optical transmission assembly to the eyelid or peri-ocular area of the patient; and
a second removable tip removably connectable to the distal end of the handpiece and configured to be positioned in contact with the eyelid or peri-ocular area of the patient to transmit the RF energy from the distal end of the RF transmission assembly to the eyelid or peri-ocular area of the patient.

2. The device as claimed in claim 1, wherein the optical transmission assembly comprises an optical fiber bundle.

3. The device as claimed in claim 1, wherein the second removable tip includes a plurality of RF electrodes to transmit the RF energy to the eyelid or the peri-ocular area of the patient.

4. The device as claimed in claim 1, wherein at least a portion of the RF transmission assembly extends externally of a cover member of the handpiece.

5. The device as claimed in claim 1, comprising said RF energy source located in at least one of: inside the handpiece or external to the handpiece.

6. The device as claimed in claim 1, wherein at least one of the first removable tip and the second removable tip is made of bio-compatible material(s).

7. The device as claimed in claim 1, wherein at least one of the first removable tip and the second removable tip is configured to be disposable.

8. The device as claimed in claim 1, wherein the first removable tip and the second removable tip are defined with a plurality of notches to facilitate assembly with the distal end of the handpiece.

9. The device as claimed in claim 1, being operable for treatment of dry eye syndrome.

10. A method of treating dry eye syndrome of a patient the method comprising:
providing a handpiece having a proximal end and a distal end;
providing a first and a second removable tip configured to be removably connectable to the distal end of the handpiece and configured to be positioned in contact with an eyelid or peri-ocular area of the patient;
connecting the first removable tip to the distal end of the handpiece;
contacting the eyelid or peri-ocular area of the patient with the first removable tip;
transmitting an intense pulse light (IPL) received from the proximal end of the handpiece to the distal end to treat the eyelid or peri-ocular area of the patient;
replacing the first removable tip with the second removable tip;
contacting the eyelid or peri-ocular area of the patient with the second removable tip;
transmitting radio-frequency (RF) energy from the proximal end of the handpiece to the distal end to treat the eyelid or peri-ocular area of the patient.

11. The method as claimed in claim 10, further comprising massaging the eyelid with the second tip during application the RF treatment.

12. The method as claimed in claim 10, comprising: performing expression on upper and lower eyelids of the eye after applying the RF treatment.

13. The method as claimed in claim 10, wherein step (a) comprises placing an eye shield on the eyeball under the eyelids, to protect the eye, prior to applying the IPL treatment.

14. The method of claim 10, being operable for treatment of dry eye syndrome.

15. The method of claim 10, wherein the second removable tip includes a plurality of RF electrodes to transmit RF energy to the eyelid or the peri-ocular area of the patient.

16. The method of claim 10, wherein transmitting of the IPL is done through a fiber bundle optical transmission assembly.

17. The method of claim 10, wherein an RF energy source located in at least one of: within the handpiece or external to the handpiece.

18. The method of claim 10, wherein at least one of the first removable tip and the second removable tip is made of bio-compatible material(s).

19. The method of claim 10, wherein at least one of the first removable tip and the second removable tip is disposable.
